SEQUENCE LISTING (43 sequences)

SEQ ID NO: 1 - DNA, 3109 bp, Homo sapiens
gggggaagtggtagcagttcctcctaactcctgccagaaacagctctcctcaacatgagagctgcacccctcctcctggccagggcagcaagccttagccttggcttcttgtttctgctttttttctggctagaccgaagtgtactagccaaggagttgaagtttgtgactttggtgtttcggcatggagaccgaagtcccattgacacctttcccactgaccccataaaggaatcctcatggccacaaggatttggccaactcacccagctgggcatggagcagcattatgaacttggagagtatataagaaagagatatagaaaattcttgaatgagtcctataaacatgaacaggtttatattcgaagcacagacgttgaccggactttgatgagtgctatgacaaacctggcagccctgtttcccccagaaggtgtcagcatctggaatcctatcctactctggcagcccatcccggtgcacacagttcctctttctgaagatcagttgctatacctgcctttcaggaactgccctcgttttcaagaacttgagagtgagactttgaaatcagaggaattccagaagaggctgcacccttataaggattttatagctaccttgggaaaactttcaggattacatggccaggacctttttggaatttggagtaaagtctacgaccctttatattgtgagagtgttcacaatttcactttaccctcctgggccactgaggacaccatgactaagttgagagaattgtcagaattgtccctcctgtccctctatggaattcacaagcagaaagagaaatctaggctccaagggggtgtcctggtcaatgaaatcctcaatcacatgaagagagcaactcagataccaagctacaaaaaacttatcatgtattctgcgcatgacactactgtgagtggcctacagatggcgctagatgtttacaacggactccttcctccctatgcttcttgccacttgacggaattgtactttgagaagggggagtactttgtggagatgcactatcggaatgagacgcagcacgagccgtatcccctcatgctacctggctgcagccccagctgtcctctggagaggtttgctgagctggttggccctgtgatccctcaagactggtccacggagtgtatgaccacaaacagccatcaaggtactgaggacagtacagattagtgtgcacagagatctctgtacaaagagtagctgccccttctcagggcagatgatgctttgagaacatactttggccattacccccagctttgaggaaaatgggctttggatgattattttatgttttagggacccccaacctcaggcaattcctacctcttcacctgaccctgcccccacttgccataaaacttagctaagttttgttttgtttttcagcgttaatgtaaaggggcagcagtgccaaaatataatcagagataaagcttaggtcaaagttcatagagttcccatgaactatatgactggccacacaggatcttttgtatttaaggattctgagattttgcttgagcaggattagataaggctgttctttaaatgtctgaaatggaacagatttcaaaaaaaaaccccacaatctagggtgggaacaaggaaggaaagatgtgaataggctgatgggcaaaaaaccaatttacccatcagttccagccttctctcaaggagaggcaaagaaaggagatacagtggagacatctggaaagttttctccactggaaaactgctactatctgtttttatatttctgttaaaatatatgaggctacagaactaaaaattaaaacctctttgtgtcccttggtcctggaacatttatgttccttttaaagaaacaaaaatcaaactttacagaaagatttgatgtatgtaatacatatagcagctcttgaagtatatatatcatagcaaataagtcatctgatgagaacaagctatttgggcacaacacatcaggaaagagagcaccacgtgatggagtttctccagaagctccagtgataagagatgttgactctaaagttgatttaaggccaggcatggtggtttacgcctataatcccagcattttgggagtccaaggtgggcagatcacttgagctcaggagctcaagatcagcctgggcaacatggtgaaaccttgtctctacataaaatacaaaaacttagatgggcatggtgctgtgtgcctatagtcccactacttgtggggctaaggcaggaggatcacttgagccccggaggtcgaggctacagcgagccaagagtgcactactgtactccagccagggcaagagagcgagaccctgtctcaataaataaataaataaataaataaataaataaataaaaacaaagttgattaagaaaggaagtataggccaggcacagtggctcacacctgtaatccttgcattttggaaggctgaggcaggaggatcactttaggcctggtgtgttcaagaccagcctggtcaacatagtgagacactgtctctaccaaaaaaaggaaggaagggacacatatcaaactgaaacaaaattagaaatgtaattatgttctaagtgcctccaagttcaaaacttattggaatgttgagagtgtggttacgaaatacgttaggaggacaaaaggaatgtgtaagtctttaatgccgatatcttcagaaaacctaagcaaacttacaggtcctgctgaaactgcccactctgcaagaagaaatcatgatatagctttgccatgtggcagatctacatgtctagagaacactgtgctctattaccattatggataaagatgagatggtttctagagatggtttctactggctgccagaatctagagcaaagccatccccgctcctggttggtcacagaatgactgacaaagacatcgattgatatgcttctttgtgttatttccctcccaagtaaatgtttgtccttgggtccattttctatgcttgtaactgtcttctagcagtgagccaaatgtaaaatagtgaataaagtcattattaggaaaaaaaaaaaaaaaaaaaaaaaaaaaaaa

SEQ ID NO: 2 - protein, 386 aa, Homo sapiens
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMHYRNETQHEPYPLMLPGCSPSCPLERFAELVGPVIPQDWSTECMTTNSHQGTEDSTD

SEQ ID NO: 3 - DNA, 60 bp, Homo sapiens
ctgtttcccccagaaggtgtcagcatctggaatcctatcctactctggcagcccatcccg

SEQ ID NO: 4 - DNA, 60 bp, Homo sapiens
aatcctatcctactctggcagcccatcccggtgcacacagttcctctttctgaagatcag

SEQ ID NO: 5 - protein, 20 aa, Homo sapiens
KELKFVTLVFRHGDRSPIDT

SEQ ID NO: 6 - protein, 20 aa, Homo sapiens
RHGDRSPIDTFPTDPIKESS

SEQ ID NO: 7 - protein, 20 aa, Homo sapiens
FPTDPIKESSWPQGFGQLTQ

SEQ ID NO: 8 - protein, 20 aa, Homo sapiens
WPQGFGQLTQLGMEQHYELG

SEQ ID NO: 9 - protein, 20 aa, Homo sapiens
LGMEQHYELGEYIRKRYRKF

SEQ ID NO: 10 - protein, 20 aa, Homo sapiens
EYIRKRYRKFLNESYKHEQV

SEQ ID NO: 11 - protein, 20 aa, Homo sapiens
LNESYKHEQVYIRSTDVDRT

SEQ ID NO: 12 - protein, 20 aa, Homo sapiens
YIRSTDVDRTLMSAMTNLAA

SEQ ID NO: 13 - protein, 20 aa, Homo sapiens
LMSAMTNLAALFPPEGVSIW

SEQ ID NO: 14 - protein, 20 aa, Homo sapiens
LFPPEGVSIWNPILLWQPIP

SEQ ID NO: 15 - protein, 20 aa, Homo sapiens
NPILLWQPIPVHTVPLSEDQ

SEQ ID NO: 16 - protein, 20 aa, Homo sapiens
VHTVPLSEDQLLYLPFRNCP

SEQ ID NO: 17 - protein, 20 aa, Homo sapiens
LLYLPFRNCPRFQELESETL

SEQ ID NO: 18 - protein, 20 aa, Homo sapiens
RFQELESETLKSEEFQKRLH

SEQ ID NO: 19 - protein, 20 aa, Homo sapiens
KSEEFQKRLHPYKDFIATLG

SEQ ID NO: 20 - protein, 20 aa, Homo sapiens
PYKDFIATLGKLSGLHGQDL

SEQ ID NO: 21 - protein, 20 aa, Homo sapiens
KLSGLHGQDLFGIWSKVYDP

SEQ ID NO: 22 - protein, 20 aa, Homo sapiens
FGIWSKVYDPLYCESVHNFT

SEQ ID NO: 23 - protein, 20 aa, Homo sapiens
LYCESVHNFTLPSWATEDTM

SEQ ID NO: 24 - protein, 20 aa, Homo sapiens
LPSWATEDTMTKLRELSELS

SEQ ID NO: 25 - protein, 20 aa, Homo sapiens
TKLRELSELSLLSLYGIHKQ

SEQ ID NO: 26 - protein, 20 aa, Homo sapiens
LLSLYGIHKQKEKSRLQGGV

SEQ ID NO: 27 - protein, 20 aa, Homo sapiens
KEKSRLQGGVLVNEILNHMK

SEQ ID NO: 28 - protein, 20 aa, Homo sapiens
LVNEILNHMKRATQIPSYKK

SEQ ID NO: 29 - protein, 20 aa, Homo sapiens
RATQIPSYKKLIMYSAHDTT

SEQ ID NO: 30 - protein, 20 aa, Homo sapiens
LIMYSAHDTTVSGLQMALDV

SEQ ID NO: 31 - protein, 20 aa, Homo sapiens
VSGLQMALDVYNGLLPPYAS

SEQ ID NO: 32 - protein, 20 aa, Homo sapiens
YNGLLPPYASCHLTELYFEK

SEQ ID NO: 33 - protein, 20 aa, Homo sapiens
CHLTELYFEKGEYFVEMYYR

SEQ ID NO: 34 - protein, 20 aa, Homo sapiens
GEYFVEMYYRNETQHEPYPL

SEQ ID NO: 35 - protein, 20 aa, Homo sapiens
NETQHEPYPLMLPGCSPSCP

SEQ ID NO: 36 - protein, 20 aa, Homo sapiens
MLPGCSPSCPLERFAELVGP

SEQ ID NO: 37 - protein, 20 aa, Homo sapiens
LERFAELVGPVIPQDWSTEC

SEQ ID NO: 38 - protein, 20 aa, Homo sapiens
VIPQDWSTECMTTNSHQGTE

SEQ ID NO: 39 - protein, 14 aa, Homo sapiens
MTTNSHQGTEDSTD

SEQ ID NO: 40 - DNA, 60 bp, Homo sapiens
aaatcagaggaattccagaagaggctgcacccttataaggattttatagctaccttggga

SEQ ID NO: 41 - protein, 20 aa, Mus musculus
ESEEFLKRLHPYKSFLDTLS

SEQ ID NO: 42 - DNA, 60 bp, Mus musculus
gaatctgaggaattcttgaagaggcttcatccatataaaagcttcctggacaccttgtcg

SEQ ID NO: 43 - protein, 32 aa, Homo sapiens
MRAAPLLLARAASLSLGFLFLLFFWLDRSVLA

(19) United States
(12) Patent Application Publication
(10) Pub. No.: US 2008/0219972 A1
(43) Pub. Date: Sep. 11, 2008

(54) Prostatic Acid Phosphatase (Pap) Materials and Methods of Use Thereof in the Prophylactic and Therapeutic Treatment of Prostate Cancer

(75) Inventors: Elena N. Klyushnenkova, Baltimore, MD (US); Richard B. Alexander, Ellicott City, MD (US)

Correspondence Address: LARCHER & CHAO LLP, P.O. BOX 1666, SKOKIE, IL, 60076, US

(73) Assignee: UNIVERSITY OF MARYLAND, BALTIMORE, Baltimore, MD (US)

(21) Appl. No.: 12/063,765

(22) PCT Filed: Aug. 16, 2006
(86) PCT No.: PCT/US2006/031961

§ 371 Date: Mar. 19, 2008

Related U.S. Application Data
(60) Provisional application No. 60/708,527, filed on Aug. 16, 2005.

(51) Int. Cl. A61K 39/395; C07H 21/00; A61K 39/00; C12N 9/14; C12N 5/06; A61P 13/00; A61K 38/46; A61K 35/12; C07K 16/18

(52) U.S. Cl. 424/130.1; 536/23.2; 424/185.1; 435/196; 424/94.6; 424/93.7; 530/387.9; 435/330

(57) ABSTRACT

A nucleic acid molecule comprising at least one nucleotide sequence encoding SEQ ID NO: 14, 15, 19, 41, or a sequence that is at least about 95% identical thereto; a composition comprising same and a method of administering same to induce an immune response; a polypeptide consisting of SEQ ID NO: 14, 15, 19, 41, or a sequence that is at least about 95% identical thereto; a composition comprising same and a method of administering same to induce an immune response; a composition comprising APC, which have been exposed to the polypeptide, and a method of administering same to treat prostate cancer; a composition comprising T-cells, which are specific for an epitope in a polypeptide consisting of SEQ ID NO: 14, 15, 19, or 41 and a method of administering same to treat prostate cancer; a composition comprising an anti-idiotypic antibody having an internal image of an epitope of a polypeptide consisting of SEQ ID NO: 14, 15, 19, or 41 and a method of administering same to treat prostate cancer; and an immortal B-cell line that produces an anti-idiotypic monoclonal antibody having an internal image of an epitope of a polypeptide consisting of SEQ ID NO: 14, 15, 19, or 41.

### CROSS REFERENCE TO RELATED APPLICATIONS

This application claims the benefit of U.S. provisional patent application No. 60/708,527, which was filed on Aug. 16, 2005, and which is hereby incorporated by reference in its entirety.

### STATEMENT OF GOVERNMENT SUPPORT

This invention was made with support from the U.S. Government under the Veterans Administration Merit Review. Therefore, the Government has certain rights in the invention.

### TECHNICAL FIELD OF THE INVENTION

The present invention relates to PAP nucleic acids and polypeptides, related compositions, and methods of use.

### BACKGROUND OF THE INVENTION

Prostate cancer is the most common malignancy among males in the U.S. It reportedly accounts for 28% of all malignancies in men. The disease is generally more aggressive in younger patients.

Although the five-year survival rates for localized prostate cancer have improved significantly, the prognosis for metastatic forms of the disease has not improved. While simple and radical prostatectomy and local radiation therapy are effective in early stages of the disease, they are of little or no benefit in later, metastatic stages of the disease. Metastatic forms of prostate cancer are generally resistant to conventional anti-neoplastic chemotherapy. The only therapy that has shown benefit in the metastatic form of prostate cancer is androgen ablation, either by castration or estrogen (diethylstilbestrol) therapy, since prostate tumor cells are typically dependent on testosterone or other androgens as growth factors. However, androgen withdrawal frequently leads to outgrowth of androgen-independent, mutant tumor cells.

Since all currently available therapies for metastatic prostate cancer are palliative at best and do not prolong survival, there remains a need for improved methods of eradicating circulating prostate tumor cells. In this regard, since the prostate is not an essential organ, one could induce an immune response to the prostate, itself, rather than to a prostate tumor, without adversely affecting the heath of the patient.

It is known that CD4 T-cells play an important role in the development of anti-tumor immune responses. The identification of naturally processed MHC class II-restricted epitopes derived from prostate differentiation antigens is critical for the development of immunotherapeutic methods for treating prostate cancer, particularly since the use of vaccines to treat cancer is well-known in the art (see, e.g., Hoover et al., Biological Therapy of Cancer, Devita, Jr., et al., eds., J. B. Lippincott Co., 1991, pp. 670-701.), including the use of compositions comprising epitopes of tumor-associated antigens or anti-idiotypic antibodies, which mimic an antigen produced by or associated with the malignant cell (see, e.g., Int'l Pat. App. Pub. No. WO 91/11465; and U.S. Pat. No. 5,053,224).

PAP is a prostate-specific differentiation antigen that is found in intracellular and secretory forms, which are identical in sequence and modification (Lin et al., J. Biol. Chem. 273: 5939-5947 (1998)). Reduced expression of the intracellular form correlates with the development of prostate cancer, and increased expression of the secretory form is associated with progression of prostate cancer from an androgen-sensitive to an androgen-resistant phase (Yeh et al., PNAS USA 96(10): 5458-5463 (1999)). In addition, full-length PAP as a prostate cancer vaccine has shown some success in clinical trials. Since PAP is only about 49% homologous with other acid phosphatases, and the homologous regions are distributed throughout the protein, PAP-specific epitopes can be identified (see, e.g., Peshwa et al., Prostate 36: 129-138 (1998); Fikes et al., U.S. Pat. App. Pub. No. 2004/0037843; and Spitler et al., U.S. Pat. App. Pub. No. 2006/0024316).

In view of the above, it is an object of the present invention to provide PAP-specific epitopes, which are strongly immunogenic in humans. Such epitopes can be used to induce an immune response. This and other objects and advantages, as well as additional inventive features, will become apparent from the detailed description provided herein.

### BRIEF SUMMARY OF THE INVENTION

The present invention provides an isolated or purified nucleic acid molecule, which comprises at least one nucleotide sequence encoding a polypeptide consisting of an amino acid sequence selected from the group consisting of SEQ ID NO: 14, SEQ ID NO: 15, SEQ ID NO: 19, SEQ ID NO: 41, an amino acid sequence that is at least about 95% identical to SEQ ID NO: 14, an amino acid sequence that is at least about 95% identical to SEQ ID NO: 15, an amino acid sequence that is at least about 95% identical to SEQ ID NO: 19, and an amino acid sequence that is a least 95% identical to SEQ ID NO: 41. The isolated or purified nucleic acid molecule is optionally part of a DNA construct comprising at least one promoter, in which case each nucleotide sequence is operably linked to a promoter, which can be the same or different.

The present invention further provides a composition comprising an above-described isolated or purified nucleic acid molecule in an amount sufficient to induce an immune response to PAP.

Still further provided is a method of inducing an immune response in a male animal. The method comprises administering to the male animal a composition comprising an above-described isolated or purified nucleic acid molecule in an amount sufficient to induce an immune response to PAP.

An isolated or purified polypeptide is also provided. The polypeptide consists of an amino acid sequence selected from the group consisting of SEQ ID NO: 14, SEQ ID NO: 15, SEQ ID NO: 19, SEQ ID NO: 41, an amino acid sequence that is at least about 95% identical to SEQ ID NO: 14, an amino acid sequence that is at least about 95% identical to SEQ ID NO: 15, an amino acid sequence that is at least about 95% identical to SEQ ID NO: 19, and an amino acid sequence that is at least about 95% identical to SEQ ID NO: 41. The polypeptide is optionally part of a fusion protein.

Also provided is a composition comprising an above-described isolated or purified polypeptide in an amount sufficient to induce an immune response to PAP.

A method of inducing an immune response in a male animal is further provided. The method comprises administering to the male animal a composition comprising an above-described isolated or purified polypeptide in an amount sufficient to induce an immune response to PAP.

Also further provided is a composition comprising antigen-presenting cells (APC). The APC have been isolated or purified from an animal, which expresses the HLA-DRB1*1501 (also referred to herein as HLA-DR2b) allele, and have been exposed to an isolated or purified polypeptide consisting of an above-described amino acid sequence (or fusion protein or conjugate thereof) or an isolated or purified nucleic acid molecule, which encodes and expresses the polypeptide (or fusion protein thereof). The APC can be dendritic cells (DC).

Accordingly, a method for the prophylactic or therapeutic treatment of prostate cancer in a male animal is provided. The method comprises administering to the male animal an effective amount of the above-described composition comprising APC. The APC can be DC.

Still further provided is a composition comprising T-cells. The composition comprises T-cells, which specifically bind to an epitope in a polypeptide consisting of an amino acid sequence of SEQ ID NO: 14, 15, 19 or 41.

Accordingly, a method for the treatment of prostate cancer in a male animal is also provided. The method comprises administering to the male animal an effective amount of the above-described composition comprising T-cells.

Even still further provided is a composition comprising an anti-idiotypic antibody. The anti-idiotypic antibody has an internal image of an epitope of a polypeptide consisting of an amino acid sequence selected from the group consisting of SEQ ID NO: 14, SEQ ID NO: 15, SEQ ID NO: 19, and SEQ ID NO: 41.

Accordingly, a method for the prophylactic or therapeutic treatment of prostate cancer in a male animal is also provided. The method comprises administering to the male animal an effective amount of the composition comprising an anti-idiotypic antibody as described above.

An immortal B-cell line that produces an anti-idiotypic monoclonal antibody having an internal image of an epitope of a polypeptide consisting of an amino acid sequence selected from the group consisting of SEQ ID NO: 14, SEQ ID NO: 15, SEQ ID NO: 19, and SEQ ID NO: 41 is also provided.

### BRIEF DESCRIPTION OF THE FIGURES

FIG. 1 is the nucleotide sequence of the coding domain sequence (CDS) of human PAP [SEQ ID NO: 1].

FIG. 2 is the amino acid sequence [SEQ ID NO: 2] encoded by the CDS of FIG. 1, except that amino acid 330 is Y, instead of H. Amino acids are referred to herein by their standard single- or three-letter notations.

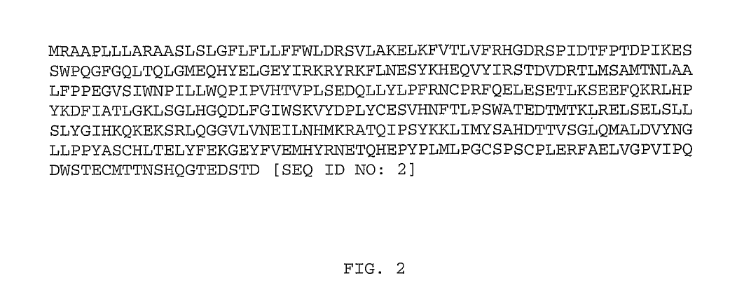

### DETAILED DESCRIPTION OF THE INVENTION

The present invention is predicated, at least in part, on the analysis of a library of overlapping 20-mer polypeptides that span the entire length of the human PAP and the discovery of a number of polypeptides, which are strongly immunogenic when injected with complete Freund's adjuvant (CFA) into transgenic mice expressing human HLA DRB1*1501 (DR2b Tg mice). Lymphocytes derived from draining lymph node (DLN) cells and spleens of such mice demonstrated strong proliferation and interferon-γ (IFN-γ) secretion in response to the same polypeptide that was used to immunize the mice. Peptide-specific T-cells also recognized whole human PAP. Therefore, human PAP epitopes, which are naturally processed and presented by antigen-presenting cells have been discovered. Polypeptides containing such epitopes stimulated an in vitro immune response by CD4 T-cells derived from peripheral blood mononuclear cells (PBMC) of human patients with granulomatous prostatitis (GP). These polypeptides and related nucleic acids, anti-idiotypic antibodies, antibodies, and APC can be used to induce an immune response to PAP.

In view of the above, the present invention provides an isolated or purified nucleic acid molecule. The nucleic acid molecule comprises at least one nucleotide sequence encoding a polypeptide consisting of an amino acid sequence selected from the group consisting of SEQ ID NO: 14, SEQ ID NO: 15 (also referred to herein as polypeptide 15), SEQ ID NO: 19 (also referred to herein as polypeptide 19), SEQ ID NO: 41, an amino acid sequence that is at least about 95% identical to SEQ ID NO: 14, an amino acid sequence that is at least about 95% identical to SEQ ID NO: 15, an amino acid sequence that is at least about 95% identical to SEQ ID NO: 19, and an amino acid sequence that is at least about 95% identical to SEQ ID NO: 41. SEQ ID NO: 41 is ESEEFLKRLHPYKSFLD TLS.

Such nucleic acid molecules can be DNA or RNA and the like, and can be synthesized (see, e.g., Oligonucleotide Synthesis, Gait, ed., 1984). Such molecules can include non-naturally occurring nucleotides/bases that encode the desired amino acid sequence. For example, the base or sugar can be methylated. In addition, the backbone of the nucleic acid molecule can be modified, e.g., a phosphorothioate backbone, methylphosphonate, methylphosphorothioate, phosphorodithioate, and combinations thereof.

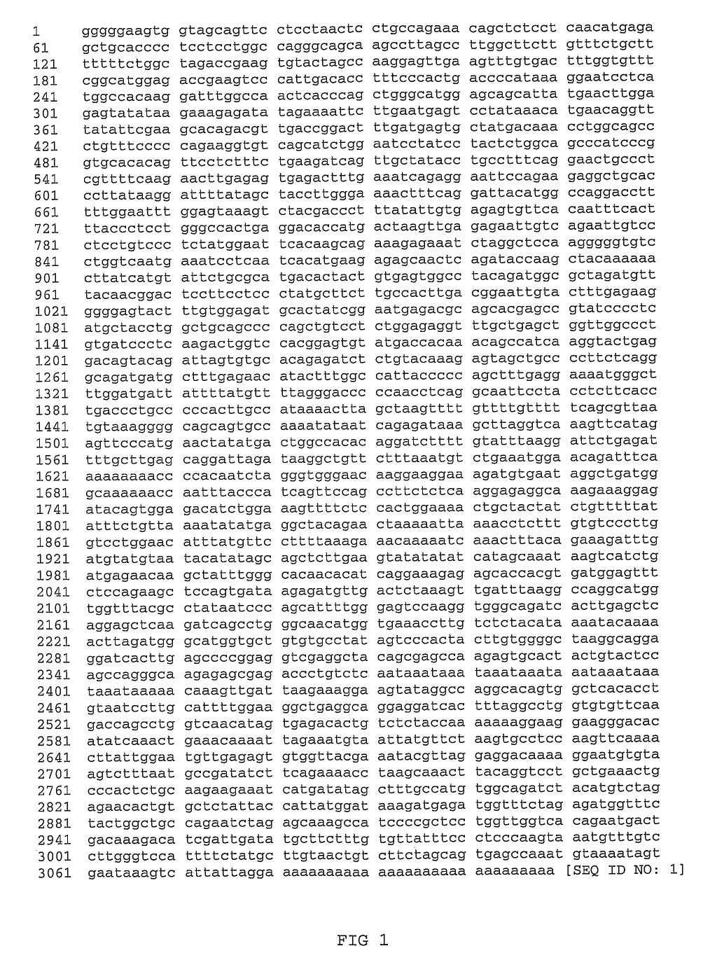

An example of a nucleotide sequence encoding the amino acid sequence consisting of SEQ ID NO: 14 is CTGTTTCCCCCAGAAGGTGTCAGCATCTGGAA TCCTATCCTACTCTGGCAGCCCATCCCG (SEQ ID NO: 3, based on GenBank Acc. No. BC016344.1; GI: 16740982, which has been reproduced herein as FIG. 1; and PNAS USA 99(26): 16899-16903 (2002)), whereas an example of a nucleotide sequence encoding the amino acid sequence consisting of SEQ ID NO: 15 is AATCCTATCCTACTCTGGCA GCCCATCCCGGTGCACACAGTTCCTCTTTCTGAAGATCAG (SEQ ID NO: 4; see FIG. 1), and an example of a nucleotide sequence encoding the amino acid sequence consisting of SEQ ID NO: 19 is AAATCAGAGGAATTCCAGAAGAGGCTGCACCC TTATAAGGATTTTATAGCTACCTTGGGA (SEQ ID NO: 40; see FIG. 1). An example of a nucleotide sequence encoding the amino acid sequence consisting of SEQ ID NO: 41 is GAATCTGAGGAATTCTTGAAGAGGCTTCATCCATATAAAAGCTTCCTGGACACC TTGTCG (SEQ ID NO: 42). One of ordinary skill in the art will appreciate, however, that due to the degeneracy of the genetic code, there are numerous other nucleotide sequences that can encode such amino acid sequences.

Such sequences can be combined to form mini-genes (see, e.g., Ihioka et al., J. Immunol. 162: 3915-3925 (199); An et al., J. Virol. 71: 2292 (1997); Thomson et al., J. Immunol. 157: 822 (1996); Whitton et al., J. Virol. 67: 348 (1993); and Hanke et al., Vaccine 16: 426 (1998)). Bi-cistronic expression vectors can be used to express the minigene and a second protein, such as a cytokine, cytokine-inducing molecule, costimulatory molecule, pan-DR binding protein, and the like.

Examples of amino acid sequences that are at least about 95% identical to SEQ ID NO: 14, SEQ ID NO: 15, SEQ ID NO: 19, or SEQ ID NO: 41 include amino acid sequences that contain one or more substitutions, insertions, additions and/or deletions. Sequence identity can be determined by aligning polypeptide sequences and applying publicly available computer algorithms, such as BLASTP (Pearson et al., PNAS USA 85: 2444-2448 (1988); Pearson, Methods Enzymol. 183: 63-98 (1990); and Altschul et al., Nucl. Acids Res. 25: 3389-3402 (1997)). The software for BLASTP is available on the FTP server of the National Center for Biotechnology Information (NCBI) or NCBI, National Library of Medicine, Building 38A, Room 8N8O5, Bethesda, Md. 20894. Once the polypeptide sequences are aligned, the number of identical amino acids over the aligned portions is identified, the number of identical amino acids is divided by the total number of amino acids of the polypeptide of interest, and the result is multiplied by 100 to determine the percentage sequence identity.

In this regard, one of ordinary skill in the art will appreciate that a fragment of a given amino acid sequence can be at least about 95% identical to the amino acid sequence. Thus, fragments are intended to be encompassed by “an amino acid sequence that is at least about 95% identical to SEQ ID NO: 14, 15, 19 or 41.” Such fragments desirably retain the immunogenicity of the full-length polypeptide. Functional fragments can be generated by mutational analysis of the polynucleotide encoding the polypeptide and subsequent expression of the resulting mutant polypeptide or by chemical/enzymatic digestion of the polypeptide, itself.

Modifications, such as substitutions, insertions, additions and/or deletions, can be introduced into the nucleic acid molecule or the polypeptide in accordance with methods known in the art (see, e.g., Adelman et al., DNA 2: 183 (1983), for oligonucleotide-directed site-specific mutagenesis). Desirably, the modification does not substantially diminish the immunogenicity of the polypeptide; rather, it is preferred that the immunogenicity remains substantially the same or increases relative to the unmodified polypeptide.

A “conservative substitution” is one in which an amino acid is substituted for another amino acid that has similar properties, i.e., similar secondary structure and hydropathic nature. Amino acid substitutions can be made on the basis of similarity in polarity, charge, solubility, hydrophobicity, hydrophilicity and/or the amphipathic nature of the residues. For example, negatively charged amino acids, such as aspartic acid and glutamic acid, can be interchanged, whereas positively charged amino acids, such as lysine and arginine, can be interchanged, and amino acids with uncharged polar head groups having similar hydrophilicity values can be interchanged. In this regard, leucine, isoleucine and valine can be interchanged, glycine and alanine can be interchanged, asparagine and glutamine can be interchanged, and serine, threonine, phenylalanine, and tyrosine can be interchanged. Other groups of amino acids that can be interchanged include: (1) ala, pro, gly, glu, asp, gln, asn, ser and thr; (2) cys, ser, tyr and thr; (3) val, ile, leu, met, ala and phe; (4) lys, arg and his; and (5) phe, tyr, trp, and his. In this regard, amino acid 14 in SEQ ID NO: 19 can be changed from D to S, amino acid 16 can be changed from I to L, and/or amino acid 17 can be changed from A to D.

The nucleic acid molecule is optionally part of a DNA construct comprising at least one promoter, in which case each nucleotide sequence is operably linked to a promoter, which can be the same or different. In addition to promoters, other control sequences, such as terminating signals and the like, can be part of the DNA construct.

For example, the nucleic acid molecule can be introduced into a suitable recombinant expression vector, such as those adapted for bacteria, such as E. coli and Salmonella typhi, yeast, such as Saccharomyces cervisiae or Pichia pastoris, or filamentous fungi, such as Aspergillus nidulans. The bacteria, yeast, or fungi can be grown in continuous culture. The polypeptide, which is produced during culture, then can be isolated and purified. Alternatively, the nucleic acid molecule can be introduced into an insect virus expression vector, such as recombinant baculovirus (e.g., Autographa californica nuclear polyhydrosis virus (AcNPV)), which, in turn, can be used to infect susceptible cultured SF9 cells, which are derived from the insect Spodotera frugiperda. Other viral vectors include vaccinia (see, e.g., U.S. Pat. No. 4,722,848), adenovirus, adeno-like virus, adeno-associated virus, retrovirus, and pox (see, e.g., Hruby, Vet. Parasitol. 29: 281-282 (1988); Uiu, “AIDS Research Reviews,” Dekker, Inc., 1991, 1: 403-416), which can be administered by a skin scratch or by injection, optionally as a liposomal formulation. Other vectors include Bacille-Calmette-Guerin (BCG; Stover et al., Nature 351: 456-460 (1991)), detoxified anthrax toxin vectors, and the like. Mammalian cells, such as Chinese hamster ovary (CHO) cells, and even plant cells can be used to express the polypeptide from the appropriate construct. One of ordinary skill in the art will appreciate that the choice of host cell will affect the nature of post-translational processing (e.g., glycosylation, folding, and the like), which, in turn, can impact the immunogenicity of the polypeptide, and subsequent purification techniques.

Alternatively, the nucleic acid molecule can behave as an effective expression system in situ when injected into an animal as “naked DNA” (see, e.g., Ulmer et al., Science 259: 1745-1749 (1993); and Cohen, Science 259: 1691-1692 (1993)). DNA delivery also can be facilitated through the use of bupivicaine, polymers, and peptides; alternatively, cationic lipid complexes, particles, or pressure (see, e.g., U.S. Pat. No. 5,922,687) can be used.

In view of the above, a composition comprising an above-described isolated or purified nucleic acid molecule in an amount sufficient to induce an immune response to PAP is provided. The amount of nucleic acid molecules in the composition can vary widely. For example, the concentration can range from less than about 0.1% to as much as about 20-50% or more by weight, usually at least about 2%.

Also provided is a method of inducing an immune response in a male animal. The method comprises administering to the male animal a composition comprising an isolated or purified nucleic acid as described above.

For example, one or more doses of the nucleic acid molecule, optionally as part of a DNA construct as described above, can be administered at bi-weekly intervals for a period of about two months. Preferred doses can range from about 0.001 mg/kg body weight to about 60 mg/kg body weight. For intravenous administration, about 1-50 mg/kg, more preferably about 5-20 mg/kg, can be used, although 1-10 mg/kg, such as 1-5 mg/kg or even 3-5 mg/kg, can be administered subcutaneously. Typically, the composition comprises a pharmaceutically acceptable carrier or excipient.

An isolated or purified polypeptide, which (i) consists of an amino acid sequence selected from the group consisting of SEQ ID NO: 14, SEQ ID NO: 15, SEQ ID NO: 19, SEQ ID NO: 41, an amino acid sequence that is at least about 95% identical to SEQ ID NO: 14, an amino acid sequence that is at least about 95% identical to SEQ ID NO: 15, an amino acid sequence that is at least about 95% identical to SEQ ID NO: 19, and an amino acid sequence that is at least abut 95% identical to SEQ ID NO: 41, and (ii) is optionally part of a fusion protein is also provided. The polypeptide can exist as a homopolymer, which comprises two or more copies of the same polypeptide, or as a heteropolymer, which comprises at least two different polypeptides.

Such polypeptides can be synthesized using standard chemical synthetic methods. Preferred methods employ commercially available solid-phase-based techniques (Merrifield, J. Am. Chem. Soc. 85: 2149-2154 (1983); and Merrifield, Science 150: 178-185 (1965)). Automated systems can be used to carry out such techniques in accordance with manufacturer's instructions. Therapeutic quantities can be recombinantly produced and purified.

The polypeptide can be modified by glycosylation or other derivatization (e.g., acetylation or carboxylation). The polypeptide also can be recombinantly produced, e.g., as part of a fusion protein, such as one that contains amino acid sequence(s) that is/are not normally found in human PAP (see, e.g., Sambrook et al., Molecular Cloning: A Laboratory Manual, 2^{nd} ed., 1989; DNA Cloning: A Practical Approach, Vols. I and II, Glover, ed.; and Perbal, A Practical Guide to Molecular Cloning (1984)). For example a nucleic acid molecule encoding the polypeptide can be ligated into an appropriate expression vector comprising a transcriptional promoter such that that nucleic acid molecule is operably linked to the promoter. Transcription termination signals, translational start sites, Kozak sequence, and stop codons also can be present in the vector. Detection and affinity purification of the expressed polypeptide can be facilitated by the presence of polynucleotide sequences in the vector encoding polypeptides, such as His⁶ or the FLAG® sequence (Sigma-Aldrich, St. Louis, Mo.).

Expression can be achieved in any appropriate host cell transformed/transfected with the expression vector. Examples of suitable host cells include, but are not limited to, those described above.

Supernatants from host/vector systems that secrete the polypeptide into culture media can be applied to a purification matrix, such as an affinity column or an ion exchange column. One or more reverse-phase HPLC steps can be employed to purify further the recombinant polypeptide.

Production of a polypeptide as a fusion protein can stabilize production. This can be accomplished by ligating polynucleotide sequences encoding two or more polypeptides into an appropriate expression vector with or without a peptidic linker. Desirably, the reading frames of the polynucleotides sequences are in phase, so that a single fusion protein that retains the biological activity of each polypeptide is produced. A peptidic linker from 1 to about 50 amino acids can be used to separate the resultant polypeptides so as to ensure that each polypeptide properly folds into its native secondary, tertiary, and quaternary structures (see, e.g., Maratea et al., Gene 49: 39-46 (1985); Murphy et al., PNAS USA 83: 8258-8262 (1986); U.S. Pat. No. 4,935,233; and U.S. Pat. No. 4,751,180). The ability to adopt a flexible extended conformation, the inability to adopt a secondary structure that could interact with functional amino acids on either one or both of the polypeptides, and the lack of hydrophobic or charged residues that might react with either one or both of the polypeptides are factors, which are taken into consideration in selecting a peptide linker. Linkers are not required when the ends of the polypeptides to be joined do not contain essential regions, such that the ends can be used to separate functional domains and prevent steric interference. Preferred peptide linker sequences contain Gly, Asn, and Ser residues. Other near neutral residues, such as Thr and Ala, also can be used.

Other additional amino acid sequence(s) can be selected to enhance the expression and/or immunogenicity of the polypeptide. For example, the polypeptide can be fused to the heavy chain of immunoglobulin G (IgG) or an APC binding protein or a dendritic cell binding protein, such as IL-D, GM-CSF, IL-1, TNF, IL-4, CD40L, CTLA4, CD28, or FLT-3 ligand. Techniques, such as the use of dehydrating agents, e.g., dicyclohexylcarbodiimide (DCCI), or the creation of linkages between sulfhydryl groups, epsilon amino groups, carboxyl groups, and the like, can be used. If desired, a cleavage site can be introduced into the fusion protein to enable separation of the polypeptide from the non-naturally occurring sequence(s). Examples of cleavage sites include a target sequence for a proteolytic enzyme or, if methionine is not present in the polypeptide, methionine, which, in turn, is cleaved by cyanogen bromide. Such methods are known in the art.

Alternatively, polypeptides can be chemically complexed with such moieties. The resulting chemical complexes are often referred to as conjugates. For example, a dehydrating agent, such as dicyclohexylcarbodiimide (DCCI) can be used to form a peptidic bond between two polypeptides. Alternatively, linkages can be formed through sulfhydryl groups, epsilon amino groups, carboxyl groups, or other reactive groups present in the polypeptide. Suitable reagents for forming such linkages are available from Pierce, Rockford, Ill. Alternatively, polypeptides can be conjugated to lipids, such as tripalmitoyl-S-glycerylcysteinylseryl-serine (P₃CSS).

The polypeptide can be expressed in situ from a suitable expression system. Any DNA construct, which is effective in producing the encoded polypeptide in the desired environment, can be used to express the polypeptide as described above.

The polypeptides can be used as reagents to evaluate an immune response, such as an immune recall response. The immune response to be evaluated can be induced by using as an immunogen any agent that can result in the production of antigen-specific CD4 T-cells, which can function as cytotoxic or helper cells. For example, PBMC samples from individuals with cancer can be analyzed for the presence of antigen-specific CTLs or HTLs using specific peptides. The response can be analyzed by tetramer staining assay, staining for intracellular lymphokines, interferon release assay, or ELISPOT assay. In this regard, the polypeptides can be used to evaluate the efficacy of a vaccine. PBMCs are obtained from an animal that has been vaccinated with an immunogen and analyzed as described. The animal is HLA typed, and polypeptides that recognize the allele-specific molecules present in the animal are selected for analysis. The immunogenicity of the vaccine is indicated by the presence of epitope-specific CTLs and/or HTLs in the PBMC sample.

The polypeptides also can be used to make antibodies. See, e.g., Current Protocols in Immunology, Wiley/Greene, N.Y.; and Antibodies: A Laboratory Manual, Harlow & Lane, Cold Spring Harbor Laboratory Press, 1989.

A composition comprising an above-described isolated or purified polypeptide in an amount sufficient to induce an immune response to PAP is also provided. The concentration of polypeptides in the composition can vary widely. For example, the concentration can range from less than about 0.1% to as much as about 20-50% or more by weight, usually at least about 2%. Fluid volume and viscosity are taken into consideration when determining the final concentration.

The composition can comprise one or more ingredients that can enhance an immune response to the polypeptide. Examples of such ingredients include adjuvants, such as CFA, incomplete Freund's adjuvant, Merk Adjuvant 65, alum, lipid A, monophosphoryl lipid A, bacteria (e.g., Bacillus-Calmette-Guerrin (BCG), Bordetella pertussis, and Mycobacterium tuberculosis), polysaccharides (e.g., glucan, acemannan, and lentinan), saponins, detoxified endotoxin (DETOX), muramyl tripeptide, muramyl dipeptide (MDP), SAF1, lipopeptides (Vitiello et al., J. Clin. Invest. 95: 341 (1995)), throglobulin, albumin, such as human serum albumin, tetanus toxoid, polyamino acids, such as poly L-lysine or poly L-glutamic acid, influenza, hepatitis B virus core protein, a lymphokine, a cytokine (e.g., IL, such as IL-1 and IL-2, IFN, such as IFN-γ, and colony stimulating factors (CSF), such as granulocyte-macrophage CSF (GM-CSF)), a cross-binding HLA class II molecule (e.g., PADRE™ (Epimmune, San Diego, Calif.; see, e.g., U.S. Pat. No. 5,736,142), nonionic block copolymers, immune-stimulating complexes (ISCOMS; Takahashi et al., Nature 344: 873-875 (1990); Hu et al., Clin. Exp. Immunol. 113: 235-243 (1998)), multiple antigen peptide systems (MAPs; Tam, PNAS USA 85: 5409-5413 (1988)), keyhole limpet hemocyanin (KLH; see, e.g., Frey et al., U.S. Pat. App. Pub. No. 2004/0241695), aluminum hydroxide, and mineral oil. Alternatively, or additionally, the composition can comprise liposomes (e.g., lipopolysaccharide (LPS), lipid A, and/or MDP; see, e.g., Liposomes, Ostro, ed., Marcel Dekker, Inc., 1983, pg. 249; Reddy et al., J. Immunol. 148: 1585 (1992); and Rock, Immunol. Today 17: 131 (1996)), and/or microspheres (e.g., poly(DL-lactide-co-glycolide) or PLG microspheres; Eldridge et al., Molec. Immunol. 28: 287-294 (1991); Alonso et al., Vaccine 12: 299-306 (1994); and Jones et al., Vaccine 13: 675-681 (1995)). If desired, polypeptides, which can be the same or different, can be coupled to a carrier, such as KLH, rotavirus VP6 inner capsid protein, pilin protein, and the like, in accordance with standard and conventional coupling techniques, optionally employing spacer moieties, to enhance immunogenicity. The composition can comprise other suitable ingredients, such as water, saline, phosphate-buffered saline, and excipients, as are known in the art.

Accordingly, also provided is a method of inducing an immune response in a male animal. Use of the term “animal” is intended to encompass humans. The method comprises administering to the male animal a composition comprising an isolated or purified polypeptide as described above.

For example, one or more doses of the polypeptide, fusion protein and/or conjugate can be administered at bi-weekly intervals for a period of about two months. Preferred doses for parenteral administration range from about 5 μg/kg body weight to about 10 mg/kg body weight or more. Typically, the composition comprises a pharmaceutically acceptable carrier or excipient.

Also provided is a composition comprising APC, which (i) have been isolated or purified from an animal, which expresses the HLA-DRB1*1501 allele, and (ii) have been exposed to an above-described isolated or purified polypeptide (or fusion protein or conjugate thereof) or an isolated or purified nucleic acid molecule, which encodes and expresses the polypeptide (or fusion protein thereof). The APC can be DC.

Following mobilization, APC and DC can be isolated from a number of tissue sources, and are conveniently isolated from peripheral blood. An example of a suitable method for the isolation of DC is disclosed in U.S. Pat. Nos. 5,976,546; 6,080,409; and 6,210,662. Briefly, buffy coats can be prepared from peripheral blood. Cells can be harvested from leukopacs, layered over columns of organosilanized colloidal silica (OCS) separation medium (prepared as described by Dorn in U.S. Pat. No. 4,927,749) in centrifuge tubes or devices. The OCS medium is preferably prepared by reacting and, thus, blocking the silanol groups of colloidal silica with an alkyl trimethoxy silane reagent. Peripheral blood mononuclear cells (PBMC) are harvested, resuspended and centrifuged to remove platelets. The resulting interface and pelleted cells are harvested and washed by centrifugation. The pellet fraction is resuspended in cell culture medium and cultured. Non-adherent cells are harvested. FACS analysis can be used to quantify the purity of DC in the interface fraction. The morphology of the cells can be evaluated using photomicroscopy. Cell-surface phenotypic analysis can be carried out through flow cytometric methods.

Enriched APC, in particular DC, can be resuspended in medium, such as RPMI medium, and incubated in the presence of a polypeptide, fusion protein or conjugate. For example, 10⁶ to 10¹¹ cells, such as 10⁷ cells, can be exposed to 100 ng/ml to 1 mg/ml of a given polypeptide, fusion protein, or conjugate. Alternatively, nucleic acid molecules are introduced into the APC or DC, such as by CaPO₄ precipitation, lipofection, naked DNA exposure, or by transfection/transformation with a viral/bacterial vector. Use of “which have been exposed to the polypeptide” is intended to encompass exposure to a polypeptide (or a fusion protein or conjugate thereof), exposure to naked DNA encoding and expressing the polypeptide (or fusion protein thereof), and transfection/transformation with a vector encoding and expressing the polypeptide (or fusion protein thereof). Primed cells are then washed and resuspended for parenteral administration, e.g., intravenous, subcutaneous, intraperitoneal, or intramuscular injection. Generally, enriched APC are administered at regular intervals for a short period of time, e.g., in bi-weekly intervals for two months or less. Doses of about 10⁷ to about 10¹¹ cells are administered. DC can be loaded in vivo by mobilizing DC and administering the above-described nucleic acid molecules and/or polypeptides.

Thus, a method for the prophylactic or therapeutic treatment of prostate cancer in a male animal is also provided. The method comprises administering to the male animal an effective amount of an above-described composition comprising APC, whereupon the male animal is treated prophylactically or therapeutically for prostate cancer.

By “prophylactic” is meant to delay the onset of prostate cancer or to prevent the onset of prostate cancer in a male animal, such as a human, which has a prostate and is at risk for developing prostate cancer. By “therapeutic” is meant to inhibit the progression, and preferably the metastasis, of prostate cancer by any degree, whether by 10%, 20%, 30%, 40%, 50% or more, or to cure the prostate cancer. Prostate cancer is considered to be cured if there is no evidence of prostate cancer or metastasis thereof for at least 1 year, preferably 2 years, more preferably 3 years, and most preferably 5 years, even if the prostate cancer recurs in the future.

Desirably, APC are isolated from the patient, primed ex vivo, and administered to the patient. Preferably, the APC are DC.

APCs primed with polypeptides, fusion proteins and/or conjugates are effective in activating T-cells to produce a cytotoxic cellular response against the polypeptide. A higher level of T-cell activation can be achieved with fusion proteins and/or conjugates.

Still further provided is a composition comprising T-cells. The composition comprises T-cells, which specifically bind to an epitope in a polypeptide consisting of an amino acid sequence of SEQ ID NO: 14, 15, 19 or 41.

Accordingly, a method for the treatment of prostate cancer in a male animal is also provided. The method comprises administering to the male animal an effective amount of the above-described composition comprising T-cells.

A composition comprising an anti-idiotypic antibody having an internal image of an epitope of a polypeptide consisting of an amino acid sequence selected from the group consisting of SEQ ID NO: 14, SEQ ID NO: 15, SEQ ID NO: 19, and SEQ ID NO: 41 is also provided.

One of ordinary skill in the art will appreciate that an anti-idiotypic antibody, which bears an internal image of an epitope, such as those described herein, can be prepared. See, e.g., Herlyn et al., Science 232: 100-102 (1986)). Methods of preparing monoclonal and polyclonal anti-idiotypic antibodies, which bear the internal image of the polypeptide, are described in U.S. Pat. No. 5,053,224, for example. Briefly, polyclonal anti-idiotypic antibodies can be produced by immunizing animals with monoclonal idiotypic antibodies raised against the polypeptide and screened for reactivity with the polypeptide and screening for antisera, which react with idiotypic antibodies to the polypeptide. Monoclonal antibodies (mAbs) also can be prepared from such animals using standard techniques of immortalizing the antibody-secreting cells of the animal and screening cultures with idiotypic antibodies in competition with the polypeptide. Human or murine mAbs are preferred, although polyclonal antibodies (pAbs), which are prepared in a variety of mammalian systems, also can be used.

A method for the prophylactic or therapeutic treatment of prostate cancer in a male animal is also provided. The method comprises administering to the male animal an effective amount of a composition comprising an anti-idiotypic antibody as described above, whereupon the male animal is treated prophylactically or therapeutically for prostate cancer.

An immortal B-cell line that produces an anti-idiotypic monoclonal antibody having an internal image of an epitope of a polypeptide consisting of an amino acid sequence selected from the group consisting of SEQ ID NO: 14, SEQ ID NO: 15, SEQ ID NO: 19, and SEQ ID NO: 41 is also provided. mAbs substantially free of other antibodies can be isolated from the supernatant of substantially pure cultures of immortal B lymphocytes. An immortal B-cell line is one, which is relatively stable and continuously produces antibodies. The cell line can be maintained in culture for several months. See, e.g., Monoclonal Antibodies, Kennett et al., 1980; Schreier et al., Hybridoma Techniques, Cold Spring Harbor Laboratory, 1980; Monoclonal Antibodies and T-Cell Hybridomas, Hammerling et al., 1981; Kozbor et al., PNAS USA 79: 6651-6655 (1982); Jonak et al., Hybridoma 2:124 (1983); Monoclonal Antibodies and Functional Cell Lines, Kennett et al., 1983; and Kozbor et al., Immunol. Today 4: 72-79 (1983)).

In view of the above, one of ordinary skill in the art will appreciate that the compositions can comprise a nucleic acid molecule, whether as naked DNA or as part of a construct, a polypeptide, optionally as part of a fusion protein, an antibody, and an anti-idiotypic antibody in various combinations. Such compositions can further comprise APC, such as DC.

Compositions as described herein can be administered by any suitable route. Parenteral routes include intracutaneous, subcutaneous, intramuscular, and intravenous injection and oral administration. The composition can be formulated appropriately, according to the route of administration. For example, the composition can be formulated in Hank's solution or Ringer's solution, along with suitable excipients providing buffering, stabilizing, and other desirable characteristics, as well as additional ingredients. See, generally, Remington's Pharmaceutical Sciences, Mack Pub. Co., Easton, Pa.

The determination of an “effective amount” and “an amount sufficient to induce an immune response” are within the ordinary skill in the art. Preferably, the amount produces a detectable immune response, such as a humoral response (circulating antibodies) or a cellular response (antigen-specific T-lymphocytes). The response may develop in days or weeks, depending on the dosage, the species or strain of animal immunized, and the immunization schedule employed. Such amounts can range from about 0.01 μg to about 100 mg per dose, such as from about 0.1 μg to about 10 mg per dose, or from about 10 μg to about 1 mg per dose. Suitable volumes for parenteral administration range from about 0.1 ml to about 5 ml.

Multiple doses can be required, such as once per week for one or two months with decreasing frequency thereafter for a period extending up to about one year. Afterwards, booster innoculations can be given every couple months for up to about five years. Depot injections may not require such a high frequency of administration.

In addition, the above compositions can be used in combination with other treatments, such as surgery. For example, a primary tumor can be surgically removed and then a vaccine can be used to slow or prevent recurrence and/or metastasis.

### EXAMPLES

The following examples serve to illustrate the present invention. The examples are not intended to limit the scope of the invention in any way.

### Example 1

This example demonstrates the immunogenicity of human PAP in DR2b transgenic mice.

DR2b transgenic (Tg) mice were injected subcutaneously with human PAP antigen (200 μg) in CFA. A proliferation assay was performed in accordance with a previously established protocol (Rich et al., Eur. J. Immunol. 34: 1251-261 (2004)) to determine if there was a specific recall response to the human PAP antigen. After nine days, DLN cells and spleens were harvested and cultured in vitro in medium containing various additives for 72 hrs. Prostate-specific antigen (PSA) was used as a negative control, whereas a purified protein derivate (PPD) of tuberculin and the mitogen concanavalin A (ConA) were used as positive controls. Cultures were pulsed with [³H]TdR, and counts per minute (CPM) were determined 18 hrs later. A robust dose-dependent immune response to human PAP was observed in the DLN cells and the spleen.

### Example 2

This example demonstrates the induction of an inflammatory immune response in the prostates of DR2b Tg mice by immunization with human PAP.

In order to determine if immunization with human PAP can induce an autoimmune inflammatory response in the prostates of DR2b Tg mice, DR2b Tg male mice were immunized subcutaneously with PAP in CFA in accordance with a previously established protocol (Rich et al., (2004), supra). A control group was injected with CFA only. Bordetella pertussis toxin (Ptx) was injected intraperitoneally on day 0 and day 2 in both groups. The frequency of PAP-reactive, EFN-γ-secreting T cells was estimated in DLN cells by ELISPOT assay (Cellular Technology Ltd., Cleveland, Ohio) on days 13 and 26 after immunization. This response correlated with the development of an inflammatory response in prostate tissue. The response to PPD served as a positive control. No response to PAP and a strong response to PPD were observed in the group injected with CFA only.

DLN cells were pooled within groups and cultured in nitrocellulose-backed 96-well plates pre-coated with capture anti-IFN-γ monoclonal antibodies (mAbs). Human PAP and PPD were added. After two days of incubation, the assay was developed, and spots were counted using an ImmunoSpot reader (Cellular Technology Ltd.). PAP-specific, IFN-γ-producing cells were detected in DLN cells of DR2b Tg mice immunized with PAP two weeks after immunization (frequency=1/5,000); the frequency increased significantly four weeks after immunization (frequency=1/1,000).

A strong antigen-specific IFN-γ response in DLN correlated with the development of an inflammatory response in prostate tissue. The level of inflammation was assessed on formalin-fixed, paraffin-embedded, H&E-stained sections four weeks after immunization.

All animals immunized with PAP developed acute and chronic subepithelial mixed inflammation focally infiltrating epithelium mostly in the dorsolateral lobes of the prostate. Three animals developed particularly strong responses. Scattered neutrophils were observed. Mice immunized with PAP also developed diffuse interstitial edema with congestion and vascular ectasia. Animals in the control group injected with only CFA+Ptx showed normal prostate structure; in some animals acute inflammation was observed predominantly in the adjacent fat tissue and coagulating gland, probably due to the Ptx injection. Thus, these data indicate that immunization with human PAP can break tolerance to the mouse antigen and induce an autoimmune response in the mouse prostate.

### Example 3

This example describes the identification of polypeptides derived from human PAP that contain epitopes recognized by CD4 T-cells in DR2b Tg mice.

A library of overlapping 20-mer polypeptides were derived from PAP in accordance with methods known in the art. The polypeptides are shown in Table 1 (below).

TABLE 1

| | |
|---|---|
| Amino Acids | Amino Acid Sequence |

| | | |
|---|---|---|
| [1-32] | MRAAPLLLARAASLSLGFLFLLFFWLDRSVLA | [SEQ ID NO: 43] |
| [33-52] | KELKFVTLVF RHGDRSPIDT | [SEQ ID NO: 5] |
| [43-62] | RHGDRSPIDT FPTDPIKESS | [SEQ ID NO: 6] |
| [53-72] | FPTDPIKESS WPQGFGQLTQ | [SEQ ID NO: 7] |
| [63-82] | WPQGFGQLTQ LGMEQHYELG | [SEQ ID NO: 8] |
| [73-92] | LGMEQHYELG EYIRKRYRKF | [SEQ ID NO: 9] |
| [83-102] | EYIRKRYRKF LNESYKHEQV | [SEQ ID NO: 10] |
| [93-112] | LNESYKHEQV YIRSTDVDRT | [SEQ ID NO: 11] |
| [103-122] | YIRSTDVDRT LMSAMTNLAA | [SEQ ID NO: 12] |
| [113-132] | LMSAMTNLAA LFPPEGVSIW | [SEQ ID NO: 13] |
| [123-142] | LFPPEGVSIW NPILLWQPIP | [SEQ ID NO: 14] |
| [133-152] | NPILLWQPIP VHTVPLSEDQ | [SEQ ID NO: 15] |
| [143-162] | VHTVPLSEDQ LLYLPFRNCP | [SEQ ID NO: 16] |
| [153-172] | LLYLPFRNCP RFQELESETL | [SEQ ID NO: 17] |
| [163-182] | RFQELESETL KSEEFQKRLH | [SEQ ID NO: 18] |
| [173-192] | KSEEFQKRLH PYKDFIATLG | [SEQ ID NO: 19] |
| [183-202] | PYKDFIATLG KLSGLHGQDL | [SEQ ID NO: 20] |
| [193-212] | KLSGLHGQDL FGIWSKVYDP | [SEQ ID NO: 21] |
| [203-222] | FGIWSKVYDP LYCESVHNFT | [SEQ ID NO: 22] |
| [213-232] | LYCESVHNFT LPSWATEDTM | [SEQ ID NO: 23] |
| [214-242] | LPSWATEDTM TKLRELSELS | [SEQ ID NO: 24] |
| [233-252] | TKLRELSELS LLSLYGIHKQ | [SEQ ID NO: 25] |
| [243-262] | LLSLYGIHKQ KEKSRLQGGV | [SEQ ID NO: 26] |
| [253-272] | KEKSRLQGGV LVNEILNHMK | [SEQ ID NO: 27] |
| [263-282] | LVNEILNHMK RATQIPSYKK | [SEQ ID NO: 28] |
| [273-292] | RATQIPSYKK LIMYSAHDTT | [SEQ ID NO: 29] |
| [283-302] | LIMYSAHDTT VSGLQMALDV | [SEQ ID NO: 30] |
| [293-312] | VSGLQMALDV YNGLLPPYAS | [SEQ ID NO: 31] |
| [303-322] | YNGLLPPYAS CHLTELYFEK | [SEQ ID NO: 32] |
| [313-332] | CHLTELYFEK GEYFVEMYYR | [SEQ ID NO: 33] |
| [323-342] | GEYFVEMYYR NETQHEPYPL | [SEQ ID NO: 34] |
| [333-352] | NETQHEPYPL MLPGCSPSCP | [SEQ ID NO: 35] |
| [343-362] | MLPGCSPSCP LERFAELVGP | [SEQ ID NO: 36] |
| [353-372] | LERFAELVGP VIPQDWSTEC | [SEQ ID NO: 37] |
| [363-382] | VIPQDWSTEC MTTNSHQGTE | [SEQ ID NO: 38] |
| [373-386] | MTTNSHQGTE DSTD | [SEQ ID NO: 39] |

In order to identify polypeptides from human PAP that contain epitopes recognized by CD4 T-cells, DR2b Tg mice were immunized subcutaneously with whole PAP in CFA. Splenocytes and DLN cells were harvested nine days later and were stimulated with overlapping 20-mer polypeptides derived from PAP. Since there was a limited number of DLN cells, the polypeptides were tested in pairs. Polypeptide pairs 13+15 and 18+19 stimulated proliferative responses in DLN and spleen. Polypeptides 14 and 15, but not 13 and 16, stimulated T-cell responses; and polypeptide 15 stimulated stronger responses. Polypeptide 19, but not 18 and 20, stimulated T-cell responses.

The polypeptide sequences were analyzed with the ProPred computer program (Imtech Corp., Denville, N.J.). The analysis revealed the presence of a nine amino acid HLA-DR1501-binding predicted core motif with moderate to low predicted binding affinity (score of about 4 out of 9.8).

When corresponding sequences of the mouse PAP were analyzed, the mouse polypeptide corresponding to human polypeptide 15 differed in only one amino acid; however, this amino acid was in the crucial P1 position. The mouse polypeptide corresponding to human polypeptide 19 revealed six amino acid substitutions, three of which are in positions that most likely do not affect the predicted binding score of the mouse homolog to HLA-DRB1*1501 and three of which are inside the predicted HLA-DRB1*1501 binding motif. Therefore, human polypeptide 19, rather than polypeptide 15, strongly induces autoimmune responses in DR2b mice. Thus, there is cross-reactivity to polypeptide 19 and its mouse homolog in mouse and human systems.

### Example 4

This example demonstrates the response to human PAP-derived polypeptides in DR2b Tg mice.

DR2b Tg mice were immunized subcutaneously with polypeptide 15 or 19 in CFA. Splenocytes and DLN cells were harvested 12 days later and cultured either in a flat-bottomed, 96-well tissue culture plate or in a nitrocellulose-backed plate coated with anti-mouse IFN-γ mAb in medium containing polypeptide 19 or whole human PAP at different concentrations. Cells cultured with polypeptide 15 or medium alone served as negative controls. Cultures were incubated for 48 hours. Proliferative response was determined by [³H]TdR incorporation. IFN-γ-secreting cells were detected using the ELISPOT assay. Splenocytes from DR2b Tg mice immunized with human polypeptide 19 recognized specific polypeptides as well as whole PAP in a broad range of antigen concentrations. There was no cross-reactivity with polypeptide 15, which was used as a negative control. Therefore, human polypeptide 19 is naturally processed from whole protein by mouse antigen-presenting cells. Mice, which were immunized with polypeptide 15, demonstrated a much stronger response to the specific polypeptide and whole PAP compared to polypeptide 19. The response was seen in a much broader range of antigen concentrations, suggesting that polypeptide 15-specific T-cells are of much higher affinity. The lower level of response to polypeptide 19 also indicates that this polypeptide may be recognized by low-affinity, auto-reactive mouse T-cells, whereas the higher level of response to polypeptide 15 indicates that it is most likely recognized as a foreign antigen.

### Example 5

This example demonstrates the development of human CD4 T-cell lines specific for PAP-derived 20-mer polypeptides.

The PAP polypeptides identified above were tested in human cultures. CD4 T-cell lines, which were specific for polypeptide 15 or polypeptide 19, were established from PBMC of HLA-DRB1*1501-positive GP patients and normal male donors by repeated stimulations with the polypeptide 19. After two rounds of in vitro stimulation, T-cells were plated at 2×10⁴ cells/well in 96-well, round-bottomed plates, and stimulated by irradiated autologous PBMC in the presence/absence of polypeptide 19 or control polypeptide 15.

No polypeptide-specific responses were detected in primary PBMC cultures by IFN-γ ELISPOT, proliferation assay, or IFN-γ ELISA. When CD4 T-cells were stimulated with polypeptide 15, cells from three of five GP patients and three of four normal male donors responded to the specific peptide in secondary cultures. No detectable secondary response to polypeptide 19 was seen in cells from GP patients or normal male donors. Further in vitro stimulation was required to demonstrate the specific response to polypeptide 19 in human cultures.

CD4 T-cells from two GP patients (Pr115 and Pr131) responded to polypeptide 19 in tertiary cultures as shown by IFN-γ ELISA; none of the cultures derived from normal donors were positive. The CD4 T-cell line from patient Pr131 demonstrated a particularly strong response to the polypeptide 19. The HLA restriction of the cell line was confirmed using an immortalized B-cell line derived from a patient with type II Bare Lymphocyte Syndrome (BLC cells) and engineered to express HLA-DRB1*1501. CD4 T-cells from patient Pr131 responded to the BLC-DR2b cell line in a peptide-specific manner; no response was observed when an antigen was presented by a wild-type BLC cell line that did not express HLA-DRB1*1501.

A CD4 T-cell line, which was developed from patient Pr131 by repeated stimulation with polypeptide 19, recognized human and mouse homolog polypeptides. A strong response to the mouse homolog was seen when the CD4 T-cell line was stimulated with irradiated autologous PBMC in a broader range of concentration, indicating that the mouse homolog has higher avidity compared to human polypeptide 19.

### Example 6

This example demonstrates that immunogenic polypeptides are naturally processed from whole PAP and presented by human cells.

DCs were prepared from PBMC by culturing CD14-enriched PBMC with recombinant human IL-4 and granulocyte macrophage colony stimulating factor (GM-CSF). Since immature, rather than mature DCs, are most effective in the endocytosis and processing of whole proteins, purified PAP was added into immature DC cultures overnight on the sixth day. DCs were harvested 18 hr later, washed extensively to remove antigen and cytokines, and mixed with T-cells. Polypeptide 19 was added directly to the T cells/DCs cultures. IFN-γ concentration in supernatants was determined after two days of stimulation by ELISA. CD4 T-cells specific to polypeptide 19 produced IFN-γ in an MHC-restricted manner in response to DCs pulsed with whole PAP. Human CD4 T-cells specific to the polypeptide 15 demonstrated a robust response to polypeptide 19; however, they failed to secrete IFN-γ in response to whole PAP. These data confirm that polypeptide 19 is naturally processed and presented by human cells. Based on such data, polypeptide 19 is expected to be useful in the development of a model of autoimmune prostatitis in DR2b mice.

All references, including publications, patent applications, and patents, cited herein are hereby incorporated by reference to the same extent as if each reference were individually and specifically indicated to be incorporated by reference and were set forth in its entirety herein.

The use of the terms “a,” “an,” “the,” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate better the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

What is claimed is:

1. An isolated or purified nucleic acid molecule, which (i) comprises at least one nucleotide sequence encoding a polypeptide consisting of an amino acid sequence selected from the group consisting of SEQ ID NO: 14, SEQ ID NO: 15, SEQ ID NO: 19, SEQ ID NO: 41, an amino acid sequence that is at least about 95% identical to SEQ ID NO: 14, an amino acid sequence that is at least about 95% identical to SEQ ID NO: 15, an amino acid sequence that is at least about 95% identical to SEQ ID NO: 19, and an amino acid sequence that is a least about 95% identical to SEQ ID NO: 41, and (ii) is optionally part of a DNA construct comprising at least one promoter, in which case each nucleotide sequence is operably linked to a promoter, which can be the same or different.

2. The isolated or purified nucleic acid molecule of claim 1, which comprises at least one nucleotide sequence encoding a polypeptide consisting of the amino acid sequence of SEQ ID NO: 19 or SEQ ID NO: 41.

3. A composition comprising an isolated or purified nucleic acid molecule of claim 1 in an amount sufficient to induce an immune response to prostatic acid phosphatase (PAP).

4. A composition comprising an isolated or purified nucleic acid molecule of claim 2 in an amount sufficient to induce an immune response to prostatic acid phosphatase (PAP).

5. A method of inducing an immune response in a male animal, which method comprises administering to the male animal a composition of claim 3, whereupon an immune response is induced in the male animal.

6. A method of inducing an immune response in a male animal, which method comprises administering to the male animal a composition of claim 4, whereupon an immune response is induced in the male animal.

7. An isolated or purified polypeptide, which (i) consists of an amino acid sequence selected from the group consisting of SEQ ID NO: 14, SEQ ID NO: 15, SEQ ID NO: 19, SEQ ID NO: 41, an amino acid sequence that is at least about 95% identical to SEQ ID NO: 14, an amino acid sequence that is at least about 95% identical to SEQ ID NO: 15, an amino acid sequence that is at least about 95% identical to SEQ ID NO: 19, and an amino acid sequence that is at least about 95% identical to SEQ ID NO: 41, and (ii) is optionally part of a fusion protein or a conjugate.

8. The isolated or purified polypeptide of claim 7, which consists of the amino acid sequence of SEQ ID NO: 19 or SEQ ID NO: 41, either one of which is optionally part of a fusion protein or a conjugate.

9. A composition comprising an isolated or purified polypeptide of claim 7 in an amount sufficient to induce an immune response to PAP.

10. A composition comprising an isolated or purified polypeptide of claim 8 in an amount sufficient to induce an immune response to PAP.

11. A method of inducing an immune response in a male animal, which method comprises administering to the male animal a composition of claim 9, whereupon an immune response is induced in the male animal.

12. A method of inducing an immune response in a male animal, which method comprises administering to the male animal a composition of claim 10, whereupon an immune response is induced in the male animal.

13. A composition comprising antigen-presenting cells (APC), which (i) have been isolated or purified from an animal, which expresses the HLA-DRB1*1501 allele, and (ii) have been exposed to an isolated or purified polypeptide of claim 7 or an isolated or purified nucleic acid molecule, which encodes and expresses the polypeptide.

14. The composition of claim 13, wherein the APC are dendritic cells.

15. A composition comprising APC, which (i) have been isolated or purified from an animal, which expresses the HLA-DRB1*1501 allele, and (ii) have been exposed to an isolated or purified polypeptide of claim 8 or an isolated or purified nucleic acid molecule, which encodes and expresses the polypeptide.

16. The composition of claim 15, wherein the APC are dendritic cells.

17. A method for the prophylactic or therapeutic treatment of prostate cancer in a male animal, which method comprises administering to the male animal an effective amount of the composition of claim 13, whereupon the male animal is treated prophylactically or therapeutically for prostate cancer.

18. The method of claim 17, wherein the APC in the composition are dendritic cells.

19. A method for the prophylactic or therapeutic treatment of prostate cancer in a male animal, which method comprises administering to the male animal an effective amount of the composition of claim 15, whereupon the male animal is treated prophylactically or therapeutically for prostate cancer.

20. The method of claim 19, wherein the APC in the composition are dendritic cells.

21. A composition comprising T-cells, which specifically bind to an epitope in a polypeptide consisting of an amino acid sequence of SEQ ID NO: 14, 15, 19 or 41.

22. The composition of claim 21, wherein the T-cells specifically bind to an epitope in a polypeptide consisting of an amino acid sequence of SEQ ID NO: 19 or 41.

23. A method for the treatment of prostate cancer in a male animal, which method comprises administering to the male animal an effective amount of the composition of claim 21, whereupon the male animal is treated for prostate cancer.

24. A method for the treatment of prostate cancer in a male animal, which method comprises administering to the male animal an effective amount of the composition of claim 22, whereupon the male animal is treated for prostate cancer.

25. A composition comprising an anti-idiotypic antibody having an internal image of an epitope of a polypeptide consisting of an amino acid sequence selected from the group consisting of SEQ ID NO: 14, SEQ ID NO: 15, SEQ ID NO: 19, and SEQ ID NO: 41.

26. The composition of claim 25, wherein the amino acid sequence is SEQ ID NO: 19 or SEQ ID NO: 41.

27. A method for the prophylactic or therapeutic treatment of prostate cancer in a male animal, which method comprises administering to the male animal an effective amount of the composition of claim 25, whereupon the male animal is treated prophylactically or therapeutically for prostate cancer.

28. A method for the prophylactic or therapeutic treatment of prostate cancer in a male animal, which method comprises administering to the male animal an effective amount of the composition of claim 26, whereupon the male animal is treated prophylactically or therapeutically for prostate cancer.

29. An immortal B-cell line that produces an anti-idiotypic monoclonal antibody having an internal image of an epitope of a polypeptide consisting of an amino acid sequence selected from the group consisting of SEQ ID NO: 14, SEQ ID NO: 15, SEQ ID NO: 19, and SEQ ID NO: 41.

30. The immortal B-cell line of claim 29, wherein the amino acid sequence is SEQ ID NO: 19 or SEQ ID NO: 41.
